(19) United States
(12) Patent Application Publication
(10) Pub. No.: US 2020/0206457 A1
(43) Pub. Date: Jul. 2, 2020

(54) Fluid Exchange Sensing Catheter

(71) Applicant: IRRAS AB, Stockholm (SE)

(72) Inventor: Lance Boling, San Jose, CA (US)

(21) Appl. No.: 16/730,317

(22) Filed: Dec. 30, 2019

Related U.S. Application Data
(60) Provisional application No. 62/786,805, filed on Dec. 31, 2018.

(51) Int. Cl. A61M 25/00; A61M 1/00

(52) U.S. Cl. CPC A61M 25/0029; A61M 2025/0002; A61M 1/0025

(57) ABSTRACT

A catheter for use in a fluid exchange system, including: an external catheter wall extending between a proximal end and a distal end of the catheter; a fluid column chamber at a distal region of the catheter, with the fluid column chamber including a plurality of fluid exchange apertures disposed in the external catheter wall; a first lumen in fluid communication with the fluid column chamber and adapted for aspirating fluid from the fluid column chamber; a second lumen in fluid communication with the fluid column chamber and adapted for irrigating fluid into the fluid column chamber; and a first sensor positioned to measure a fluid pressure within a fluid column of the fluid column chamber, with the first sensor disposed at a distal end of a lead, and the lead extending through a first dedicated lumen formed within the catheter or on an exterior of the catheter.

### CROSS REFERENCE TO RELATED APPLICATIONS

This application claims the benefit of U.S. Provisional Patent Application No. 62/786,805, filed Dec. 31, 2018, the disclosure of which is incorporated herein in its entirety by reference.

### BACKGROUND OF THE INVENTION

### Field of the Invention

The present disclosure relates, generally, to catheter systems and, more particularly, to a sensing catheter for use in fluid exchange applications, such as managing intracranial pressure and cerebral spinal fluid drainage.

### Description of Related Art

There are many kinds of catheters which are used for fluid infusion and aspiration in a clinical or preclinical setting. Traditionally, the catheter includes a first end that is inserted in biological material, referred to as the “distal” end, and a second end that remains outside the biological material, referred to as the “proximal” end.

Most of existing catheters have a single lumen and through this lumen the user can alternatively infuse or aspirate liquids. For example, in a clinical setting, the common intravenous catheter either aspirates blood samples—usually immediately after it's insertion to the vein—or infuses solutions of drugs and, or, nutrients—usually for many hours or days following insertion.

More recently, concurrent fluid exchange catheters have been developed. An example of one type of concurrent fluid exchange catheter is disclosed in U.S. Pat. No. 8,398,581, the entire contents of which are incorporated herein by reference. In certain embodiments, this fluid exchange catheter includes an outer lumen for aspiration and an inner lumen for infusion where the distal end of the inner lumen is disposed within an interior lumen space of the outer lumen.

Fluid exchange catheters having sensing capabilities have also been described in the art. These sensing capabilities can aid in the ability of these catheters to monitor the surrounding tissue, and can also improve upon the fluid exchange process. An example of a fluid exchange catheter having such sensing capabilities is described in, for example, United States Patent Application Publication No. 2016/0375221, the entire contents of which are incorporated herein by reference.

### SUMMARY OF THE INVENTION

In one example of the present disclosure, a catheter for use in a fluid exchange system may include an external catheter wall extending between a proximal end and a distal end of the catheter, a fluid column chamber at a distal region of the catheter, wherein the fluid column chamber comprises a plurality of fluid exchange apertures disposed in the external catheter wall, a first lumen in fluid communication with a fluid column in the fluid column chamber and adapted for aspirating fluid from the fluid column chamber, a second lumen in fluid communication with the fluid column chamber and adapted for irrigating fluid into the fluid column chamber, and a first sensor positioned to measure a fluid pressure within the fluid column chamber, wherein the first sensor is disposed on a lead, the lead being provided within an interior of the catheter or on an exterior of the catheter.

In another example of the present disclosure, the first sensor may be configured to gather data that can be used to perform at least one of the following functions: monitor fluid pressure, adjust a supply of the fluid to a patient and adjust a drainage of the fluid from the patient. The first sensor may be affixed at its distal end to an inner surface of the fluid column chamber. A second dedicated lumen may be provided. A distal end of the second dedicated lumen may open into a sensing channel. The sensing channel may be in fluid communication with an area external to the catheter. A second sensor may be positioned to measure a fluid pressure within the sensing channel. The second sensor may be positioned at a distal end of a lead, the lead extending through the second dedicated lumen. The second sensor may measure a fluid pressure external to the catheter and a fluid pressure in the fluid column chamber. The first sensor may be provided on the lead with at least one additional sensor. A second sensor may be provided on an exterior surface of the external catheter wall. An aperture may be defined in the external catheter wall such that at least a portion of the second sensor is provided within the aperture. The first sensor may be adhesively affixed to the catheter.

In another example of the present disclosure, a fluid exchange system may include a control unit including a processor; a tube set attachment removably connected to the control unit, the tube set attachment including a tube set fluidly connected to a fluid source and a drainage receptacle; a catheter fluidly connected to the tube set, the catheter including an external catheter wall extending between a proximal end and a distal end of the catheter; a fluid column chamber at a distal region of the catheter, wherein the fluid column chamber comprises a plurality of fluid exchange apertures disposed in the catheter wall; a first lumen in fluid communication with the fluid column chamber and adapted for aspirating fluid from the fluid column chamber; a second lumen in fluid communication with the fluid column chamber and adapted for irrigating fluid into the fluid column chamber; and a first sensor positioned to measure a fluid pressure within a fluid column of the fluid column chamber, wherein the sensor is disposed on a lead, the lead extending through a first dedicated lumen formed within the catheter or on an exterior of the catheter, wherein the control unit is configured to supply the fluid to a patient through the tube set and drain the fluid from the patient via the tube set, and wherein the control unit is configured to receive measurements from the first sensor to monitor fluid pressure, adjust the supply of the fluid to the patient, or adjust the drainage of the fluid from the patient.

In another example of the present disclosure, the first sensor may be configured to gather data that can be used to perform at least one of the following functions: adjust a supply of the fluid to a patient and adjust a drainage of the fluid from the patient. The first sensor may be affixed at its distal end to an inner surface of the catheter. A second dedicated lumen may be provided. A distal end of the second dedicated lumen may open into a sensing channel. The sensing channel may be in fluid communication with an area external to the catheter. A second sensor may be positioned to measure a fluid pressure within the sensing channel. The second sensor may be positioned at a distal end of a lead, the lead extending through the second dedicated lumen. The first sensor may measure a fluid pressure external to the catheter and a fluid pressure in the fluid column chamber. The first sensor may be provided on the lead along with at least one additional sensor. A second sensor may be provided on an exterior surface of the external catheter wall. An aperture may be defined in the external catheter wall such that at least a portion of the second sensor is provided within the aperture. The first sensor may be adhesively affixed to the catheter.

In another example of the present disclosure, a computer-implemented method of using a fluid exchange system may include initiating, using a processor, a control unit to deliver a fluid through the second lumen; receiving, at the processor, a first pressure value of the fluid, wherein the first pressure value is measured by the first sensor; initiating, using the processor, the control unit to deliver additional fluid through the second lumen; receiving, at the processor, a second pressure value of the fluid, wherein the second pressure value is measured by the first sensor; comparing, using the processor, the first pressure value and the second pressure value; and in an event a difference between the first and second pressure values exceeds a pressure threshold value, initiating, using the processor, the control unit to drain the fluid through the first lumen, and, in the event the difference between the first and second pressure values is less than the pressure threshold value, initiating, using the processor, the control unit to deliver additional fluid through the second lumen.

In another example of the present disclosure, the first pressure value and the second pressure value may be received from the first sensor provided at at least one of an exterior surface of the catheter and a position in the fluid column chamber defined in the catheter. The first pressure value and the second pressure value may be received from the first sensor provided at an exterior surface of the catheter and a second sensor provided at a position in the fluid column chamber defined in the catheter. The method may further include detecting, using the processor, an insertion pressure value for the catheter using the first sensor.

In another example, provided is a catheter for use in a fluid exchange system, comprising: an external catheter wall extending between a proximal end and a distal end of the catheter; a fluid column chamber provided within the catheter, wherein the fluid column chamber comprises a plurality of fluid exchange apertures disposed in the external catheter wall; a first lumen in fluid communication with the fluid column chamber and adapted for aspirating fluid from the fluid column chamber; a second lumen in fluid communication with the fluid column chamber and adapted for irrigating fluid into the fluid column chamber; and a lead positioned either internally or externally to the catheter, the lead comprising a plurality of sensors positioned in intervals along the length of the lead, wherein the sensors are configured to measure a fluid pressure either within the fluid column chamber or external to the catheter to identify a fluid pressure gradient along a length of at least a portion of the catheter.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a side view of a fluid exchange sensing catheter according to one aspect of the present disclosure;

FIG. 2 is a cross-sectional view of the fluid exchange sensing catheter of the type shown in FIG. 1, illustrating a multi-lumen construction for fluid management and for accommodating sensors according to one aspect of the present disclosure;

FIG. 3 is a cross-sectional view of a prior art fluid exchange catheter, included for comparison purposes to highlight improvements in the fluid exchange sensing catheter of FIG. 2 according to aspects of the present disclosure;

FIG. 4 a perspective view of an optical pressure sensing system for use with the fluid exchange sensing catheter of FIG. 1 according to an aspect of the present disclosure;

FIG. 5 is a perspective view of the fluid exchange sensing catheter of the type shown in FIG. 1, illustrating exemplary sensors in phantom according to one aspect of the present disclosure;

FIG. 6 is a partial cut-away view of the fluid exchange sensing catheter of the type shown in FIG. 1, illustrating exemplary sensors according to one aspect of the present disclosure;

FIG. 7 is a sectional view of the fluid exchange sensing catheter of the type shown in FIG. 1, illustrating exemplary sensors according to one aspect of the present disclosure;

FIG. 8 is an alternate design of a fluid exchange sensing catheter of the type shown in FIG. 1, illustrating a variety of additional sensors on multi-sensor leads in a variety of locations according to aspects of the present disclosure;

FIG. 9 is an enlarged view of an optical pressure sensor for use with the fluid exchange sensing catheter according to an aspect of the present disclosure, including the input optical signal (Input) and resulting output waveform (Output); and

FIG. 10 is a graph illustrating exemplary pressure readings from the fluid exchange sensing catheter of the type shown in FIG. 1 according to one aspect of the present disclosure.

### DESCRIPTION OF THE INVENTION

For purposes of the description hereinafter, the terms “upper”, “lower”, “right”, “left”, “vertical”, “horizontal”, “top”, “bottom”, “lateral”, “longitudinal”, and derivatives thereof, shall relate to the disclosed apparatus as it is oriented in the figures. However, it is to be understood that the apparatus of the present disclosure may assume alternative variations and step sequences, except where expressly specified to the contrary. It is also to be understood that the specific systems and processes illustrated in the attached drawings, and described in the following specification, are simply exemplary examples of the apparatus disclosed herein. Hence, specific dimensions and other physical characteristics related to the examples disclosed herein are not to be considered as limiting.

As used herein, the terms “communication” and “communicate” refer to the receipt, transmission, or transfer of one or more signals, messages, commands, or other types of data. For one unit or device to be in communication with another unit or device means that the one unit or device is able to receive data from and/or transmit data to the other unit or device. A communication may use a direct or indirect connection, and may be wired and/or wireless in nature. Additionally, two units or devices may be in communication with each other even though the data transmitted may be modified, encrypted, processed, routed, etc., between the first and second unit or device. It will be appreciated that numerous arrangements are possible. Any known electronic communication protocols and/or algorithms may be used such as, for example, UDP, TCP/IP (including HTTP and other protocols), WLAN (including 802.11 and other radio frequency-based protocols and methods), analog transmissions, cellular networks, and/or the like.

Illustrative embodiments of the disclosure are described below. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present disclosure is directed to a sensing catheter for use in any of a variety of fluid exchange applications, including but not limited to those shown and described in International Patent Application Publication No. WO/2018/167740, which is incorporated herein in its entirety by reference. As will be described below, the fluid exchange sensing catheter of the present disclosure may include any of a variety of sensing capabilities to aid in the functionality of the catheter beyond fluid exchange. For example, the additional sensors may measure any of a variety of physiological parameters, including but not necessarily limited to, pressure, differential pressure, acoustic, stress, displacement, temperature, vibration, biochemical, chemical, electrical properties (e.g. impedance, polarity, potential), any combination of these physiological parameters, and/or any combination of additional physiological parameters based on additional sensor technologies or capabilities now in existence (e.g. MEMS micro-sensors, nano-surfaces, Wheatstone bridges, optical sensors, electrical sensors) and/or developed in the future.

The sensors can be configured, for example, to compare measurements, to differentiate measurements along the catheter length or between its various surfaces, or to take different types of measurements. In the case of fluid exchange, the sensors can be configured to collect therapeutic protocol/process data on infusion and aspiration of fluids, including pressure, flow, pressure wave form, intracranial reaction, and blockage or obstruction of flow. Accordingly, the sensors can provide feedback to a controller or user. The sensors can be used to collect data on pulse and respiration, for example, and this data can be used to compare baseline and therapeutic response in a patient. This type of data is useful to assessing injury to tissues, reperfusion of tissues and responsiveness of tissues. The sensors can also be used to detect catheter insertion pressures against anatomy to achieve the desired tissue apposition, avoid tissue structural damage, or assist in catheter navigation to avoid tissue contact and/or damage/injury.

The sensors may be positioned anywhere along the length of the fluid exchange catheter, including but not limited to a distal region, and may be incorporated into and/or in communication with the catheter in any suitable manner, including but not limited to: 1) integrally formed into the walls of the catheter structure (e.g. via laminating), 2) disposed within dedicated lumens formed in the walls of the catheter structure, 3) disposed within and/or extending into one or more fluid exchange lumens of the catheter structure; and/or 4) any combination of the above. Moreover, the sensor technologies may be positioned and/or configured to sense physiological parameters within the catheter and/or external to the catheter without departing from the scope of the disclosure. The sensors may be coated by a flexible adhesive both to fasten the sensor to a lead in a desired location and/or to create a protective membrane or pressure-reactive diaphragm over any or all of the sensors.

While the fluid exchange sensing catheter is described below within the context of pressure sensing and, more specifically, optical pressure sensing, it will be appreciated that the embodiments shown herein and the use of optical pressure sensing technology is set forth by way of example only and not limiting as to the fundamental concept of providing a sensing catheter for fluid exchange applications.

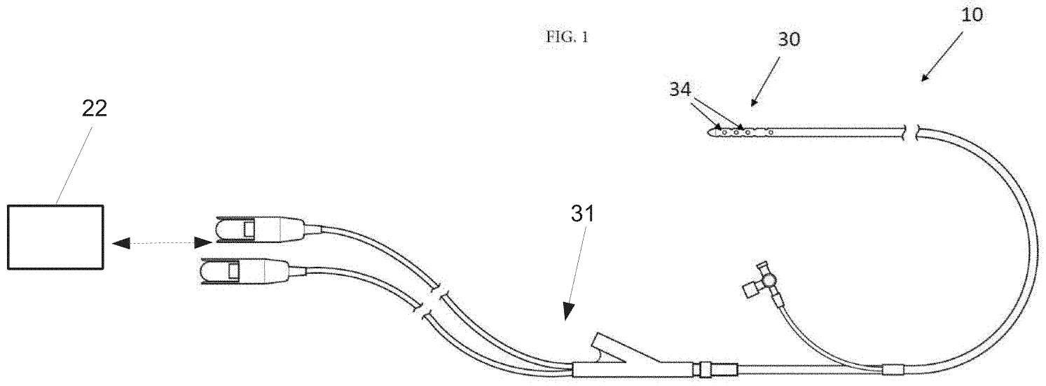

FIG. 1 shows a fluid exchange sensing catheter 10 (hereinafter “sensing catheter 10”) according to one aspect of the present disclosure. The sensing catheter 10 has a distal region 30 equipped with a plurality of fluid exchange apertures 34. The sensing catheter 10 may be dimensioned and configured for use with any of a variety of fluid exchange systems, including but not limited to the fluid exchange system of the type shown and described in International Patent Application Publication No. WO/2018/167740, the disclosure of which is incorporated herein in its entirety by reference. As will be described below, the sensing catheter 10 is advantageous in that it may include any of a variety of sensing capabilities to aid in the functionality of the catheter beyond mere fluid exchange. The sensing catheter 10 may be fluidly connected to a tube set 31 that is used to supply fluid to a patient and/or drain fluid from the patient. The details of the use of this type of tube set 31 is described in further detail in International Patent Application Publication No. WO/2018/167740. Furthermore, a control unit 22 may be operatively connected to the tube set 31 and is operated to adjust the supply and/or drainage of the fluid to/from the patient based on measurements taken using sensors provided in the sensing catheter 10.

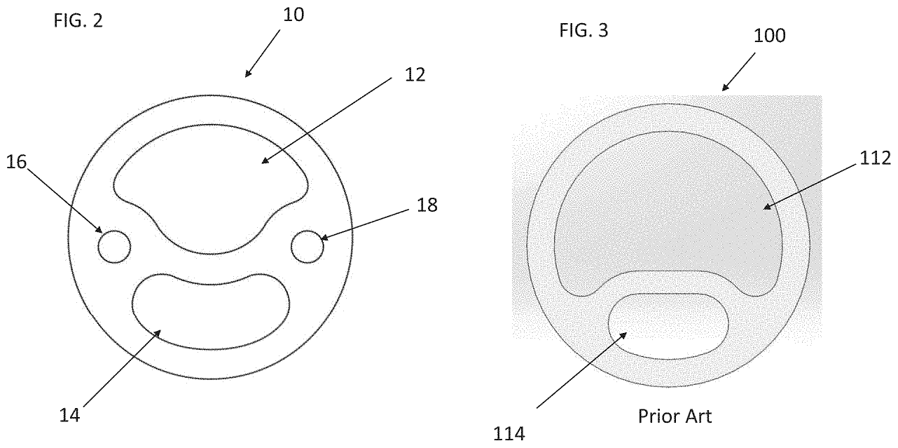

FIG. 2 shows a cross-sectional view of a sensing catheter 10 according to one aspect of the present disclosure. With reference to FIG. 2, the sensing catheter 10 may be a multi-lumen catheter defined by an external catheter wall (33). Contained within the external catheter wall can be at least one lumen 12 for aspiration, at least one lumen 14 for irrigation, and one or more dedicated lumens 16, 18 dimensioned to receive a lead having one or more sensors for sensing any of variety of physiological parameters according to the present disclosure. The lumens 12, 14, 16, 18 can be defined by one or more lumen walls, one or more of which may be the external catheter wall (33). By way of comparison, a prior art fluid exchange catheter 100 is shown in FIG. 3, and includes aspiration lumen 112 and irrigation lumen 114. When compared, it will be apparent that the aspiration lumen 12 and irrigation lumen 14 of the sensing catheter 10 of FIG. 2 are different from the aspiration lumen 112 and irrigation lumen 114 of the prior art fluid exchange catheter 110 in FIG. 3 in multiple respects. First, the aspiration lumen 12 and irrigation lumen 14 are closer in volume to one another (e.g., the volumes are within at least 25% of one another), which serves to more closely balance the irrigation and aspiration flow during use. Second, the wall structure of the sensing catheter 10 is more resistant to kinking and allows for greater rotational consistency during placement and use.

The sensing catheter 10 may be used for the delivery of drugs or therapeutic agents and their antidotes for thrombolysis, coagulation, chemotherapy, infection management, hormone therapy, cell seeding, cell therapy, markers, and/or therapies applied directly to the targeted pathology and its surrounding tissue in a patient. Delivery of such agents may also be directed to the fluid within the sensing catheter 10 for purposes of mixing, dissolving or changing the character of infused or aspirated fluids. Generally, such drugs are not particularly limited to any category of pharmaceutical fluids. Drugs suitable for catheter administration are generally known to the skilled person, such as all eligible drugs for local infusion under the skin. At least one drug or several different drugs is/are selected from the group including antibiotics, anti-inflammatory drugs (e.g. corticosteroids, immune selective anti-inflammatory drugs, etc.), analgesics (e.g. non-steroidal anti-inflammatory drugs, opioids, etc.), chemotherapeutic drugs (e.g. alkylating agents, antimetabolites, anthracyclines, etc.), and hormones (e.g. insulin, HGH, etc.). The sensing catheter 10 of the present disclosure can also be used in the treatment of pain. Accordingly, in particular aspects, the (at least one) drug is selected from analgesics. Analgesics as used herein may include narcotics or the like.

In another embodiment, the sensing catheter 10 of the present disclosure is used in the treatment of cancer. Non-limiting examples for cancer include a pancreatic tumor, a liver tumor and a brain tumor, such as glioma or craniopharyngioma. Accordingly, in particular embodiments, the (at least one) drug is selected from chemotherapeutic drugs, such as from cytostatic and cytotoxic chemotherapy drugs. Non-limiting example for such drugs include fluorouracil, methotrexate, purine analogs, nitrosoureas, platinum compounds, alkylating agents, antitumor antibiotics, etc.

In particular aspects, the sensing catheter 10 of the present disclosure is used for the removal of substances, such as undesired substances, from the body. Preferred examples of such substances are selected from the group including blood, coagulated blood, blood clot(s) (thrombus/thrombi), pus, toxic substance(s), superfluous drug(s), and/or pathological tissue(s). Other examples of such substances include tissue, such as tissue sample(s).

In one aspect, the sensing catheter 10 of the present disclosure is used in the treatment of cerebral vasospasm. In particular aspects, the sensing catheter 10 of the present disclosure is used in the treatment of subarachnoid hemorrhage (SAH). The latter aspects may involve the clearing of subarachnoid blood and/or administration with the administration of at least one drug. Preferred non-limiting examples for such drugs are papaverine, urokinase, rTPA, etc. In one aspect, the sensing catheter 10 of the present disclosure is used as a self-regulating system, such as a self-regulating system not requiring the presence of a clinician, doctor and/or medical personnel, or a self-regulating system exceeding human capabilities as regards e.g. (rapid) treatment changes. In particular aspects, the sensing catheter 10 of the present disclosure is used in an intensive care unit (ICU). In particular aspects, the sensing catheter 10 of the present disclosure is used for monitoring a site within a patient's body, which monitoring may (by way of example only) include observation (direct and/or via closed circuit or other viewing technologies) and/or video-recording.

Flow control within the sensing catheter 10 may be desired to provide a specified flow protocol, manage infusion and aspiration flow, or optimize the effect of infusion flow without loss of infused fluids to the aspiration tract, for example.

The sensing catheter 10 will be described below, by way of example only, within the context of pressure sensing and, more specifically, optical pressure sensing, but this is only a representative example and not limiting as to the fundamental concept of providing a sensing catheter for fluid exchange applications.

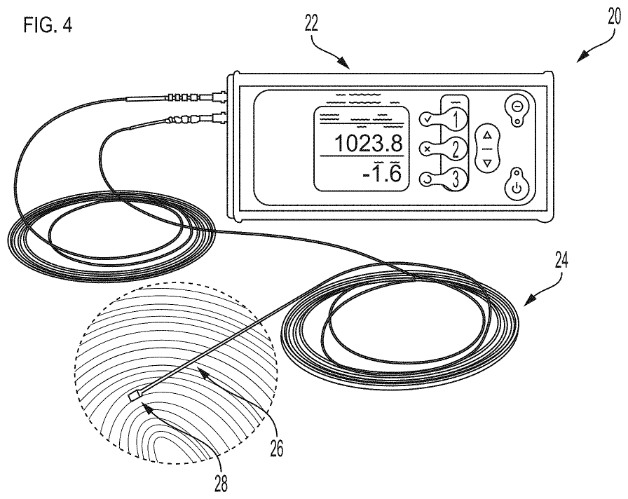

FIG. 4 shows an optical pressure sensing system 20 for use with the sensing catheter 10. The optical pressure sensing system 20 includes a control unit 22 and one or more fiber optic cables 24 each including a lead 26 equipped with one or more optical pressure sensors 28. The control unit 22 may include a processor to initiate and conduct the processes discussed below. As will be described in greater detail below, the control unit 22 is configured to emit light which travels through the fiber optic cables 24 and lead 26 into the optical sensor 28, at which point the light is reflected back to the control unit 22. Depending upon the pressure being exerted upon the optical pressure sensor 28 by the physiological input (e.g. column pressure within the sensing catheter 10 and/or intracranial pressure outside the sensing catheter 10), the control unit 22 will determine the pressure by analyzing the reflected light.

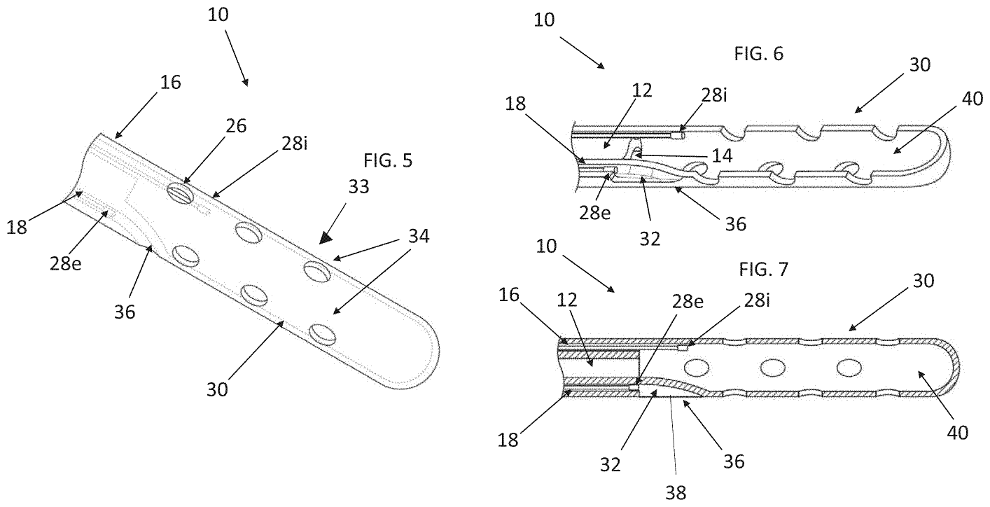

FIGS. 5-7 illustrate, by way of example only, one manner in which the optical pressure sensors 28 of FIG. 4 may be provided in communication with the sensing catheter 10 to measure pressure inside and outside the sensing catheter 10 according to an aspect of the disclosure. To so do, a first optical pressure sensor 28i (for “internal”) is dimensioned to pass through the dedicated lumen 16 and reside within a fluid column chamber 40 of the sensing catheter 10, which may receive a fluid column, while a second optical pressure sensor 28e (for “external”) is dimensioned to reside within a distal region of the dedicated lumen 18 adjacent to a sensing channel 32 formed along the exterior of the sensing catheter 10. In one example, a fluid column may be interpreted as the fluid that travels through one or more of the lumens (including irrigation and drainage) of a catheter.

The internal pressure sensor 28i is configured to sense the pressure of the fluid column within the sensing catheter 10 by virtue of being positioned inside the fluid column chamber 40 formed within the distal region 30 of the fluid exchange catheter 10. This allows the internal pressure sensor 28i to dynamically monitor the pressure in the fluid column chamber 40 during use, such as due to the cyclical and/or intermittent irrigation and/or aspiration for fluid management purposes. In one example of the present disclosure, the internal pressure sensor 28i measures a fluid pressure in the fluid column chamber 40. Although the internal pressure sensor 28i is shown extending a given distance from the dedicated lumen 16, it will be appreciated that this is merely an example and that the internal pressure sensor 28i may be positioned at any suitable location within the fluid column chamber 40 without departing from the scope of the disclosure. For example, in certain instances it may be preferred or optimal to position the internal pressure sensor 28i at one of the following positions: a) adjacent to the distal end of the fluid column chamber 40 (that is, away from the irrigation lumen 14) in order to focus on aspiration pressure; b) adjacent to the irrigation lumen 14 in order to focus on irrigation pressure; and c) at a mid-point location in order to determine the fluid column pressure as influenced by both aspiration and irrigation. All are within the scope of the present disclosure, as is the feature of dynamically changing the location of the internal pressure sensor 28i during use based on any of a variety of system and/or clinician feedback and/or input. Using the pressure measured by the internal pressure sensor 28i, this information can be used, for example, to determine whether the supply of fluid to a patient's brain and/or drainage of fluid from the patient's brain must be adjusted. The present system may be used in treating, for example, TBI (traumatic brain injuries), IVH (inter-ventricular hemorrhage), cSDH (chronic Subdural Hemorrhage, and Ventriculitis), but it is to be understood that this measurement is valuable when treating other conditions, such as any condition where irrigation and/or drainage occur.

The external pressure sensor 28e is configured to sense the pressure outside the sensing catheter 10 by virtue of being positioned adjacent the sensing channel 32, which itself may be configured to enable direct pressure and indirect pressure measurements. In this embodiment, sensing channel 32 is disposed at the distal end of dedicated lumen 18 such that the distal end of dedicated lumen 18 opens into sensing channel 32. In this configuration, both external pressure sensor 28e and sensing channel 32 are isolated from internal pressure sensor 28i and fluid column chamber 40 through a lumen wall of dedicated lumen 18. Direct pressure measurement is enabled by providing a side opening 36 along the exterior surface of the sensing catheter 10 that is in open fluid communication with sensing channel 32 such that the external pressure sensor 28e is directly exposed to the environment outside the sensing catheter 10. Indirect pressure measurement is enabled by providing a membrane 38 over the side opening 36 such that the membrane 38 transfers pressure-induced displacement from the outside environment indirectly to the sensing channel 32 and external pressure sensor 28e. The membrane 38 may be any of a variety of suitable materials and/or thicknesses in order to optimize the pressure-indicted displacements to the external pressure sensor 28e. In either event, the external pressure sensor 28e enables the measurement of pressure in the environment outside the sensing catheter 10, such as (by way of example only) intracranial pressure in deep brain hemorrhagic stroke. Using the measured pressure in the environment outside the sensing catheter 10, this information can be used to determine whether the supply of fluid to a patient's brain and/or drainage of fluid from the patient's brain must be adjusted.

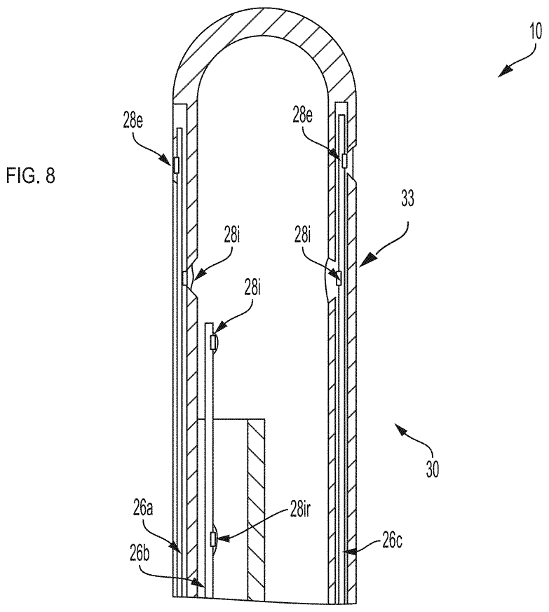

FIG. 8 illustrates an alternative configuration and arrangement of sensing catheter 10. The configuration of FIG. 8 illustrates that there may be more than one internal optical pressure sensor 28i and/or external optical pressure sensor 28e without departing from the scope of the present disclosure, and that there may be more than one sensor 28 disposed along each lead 26. By way of example only, the sensing catheter 10 may be equipped to receive multiple leads 26a, 26b, 26c, wherein each lead 26a, 26b, 26c includes multiple sensors. Lead 26a, for example, is disposed in a lumen formed in the wall of the distal region 30 (e.g., dedicated lumen 16) and includes at least one internal pressure sensor 28i (exposed to the fluid column chamber 40 via an internal side opening in the lumen) and at least one external pressure sensor 28e (exposed to the outside environment via an external side opening in the lumen). Lead 26b, for example, is disposed within and extending from the irrigation lumen 14 and includes at least one internal pressure sensor 28i (positioned to float freely within the fluid column chamber 40) and at least one irrigation pressure sensor 28ir (for “irrigation”) positioned within the irrigation lumen 14. Lead 26c, for example, much like lead 26a is disposed in a lumen formed in the wall of the distal region 30 (e.g., dedicated lumen 18) and includes at least one internal pressure sensor 28i (exposed to the fluid column chamber 40 via an internal side opening in the lumen) and at least one external pressure sensor 28e (exposed to the outside environment via an external side opening in the lumen). The internal pressure sensor 28i may be configured to measure a fluid pressure value of the fluid in the fluid column chamber 40. The external pressure sensor 28e may be configured to measure a fluid pressure value of the fluid external to the sensing catheter 10.

In one example, one of the leads 26a may include multiple sensors. For example, lead 26a can be disposed in a lumen formed in the wall of the distal region 30 (e.g., dedicated lumen 16) and includes at least one internal pressure sensor 28i (exposed to the fluid column chamber 40 via an internal side opening in the lumen) and at least one external pressure sensor 28e (exposed to the outside environment via an external side opening in the lumen). In one example, the sensors 28i, 28e may be used to measure a fluid pressure gradient for the fluid passing through the fluid column chamber 40 and/or external to the sensing catheter 10. In one example, each sensor 28i, 28e may take a fluid pressure measurement of the fluid passing through the fluid column chamber 40 and/or external to the sensing catheter 10 and the fluid pressure measurements may be compared to one another to measure a pressure gradient or difference in pressure along the sensing catheter 10. During insertion of the sensing catheter 10 into a patient's vasculature, the fluid pressure within and/or external to the sensing catheter 10 may vary. Therefore, by providing one or more sensors 28i, 28e along the length of sensing catheter 10, the sensors 28i, 28e can provide fluid pressure feedback to the control system to adjust or alter the insertion of the sensing catheter 10 into the patient, if needed. Similarly, the sensors 28i, 28e may be used to measure a fluid pressure gradient within and/or external to the sensing catheter 10 after the sensing catheter 10 has been inserted into the patient and, in a particular example, when the sensing catheter 10 has been inserted into specific regions of the patient's brain. It is also to be understood that the sensors 28i, 28e may be used to measure a compliance of the sensing catheter 10 as fluid is directed through the fluid column chamber 40. Compliance is understood to describe how the brain is compressed or relaxed/expanding.

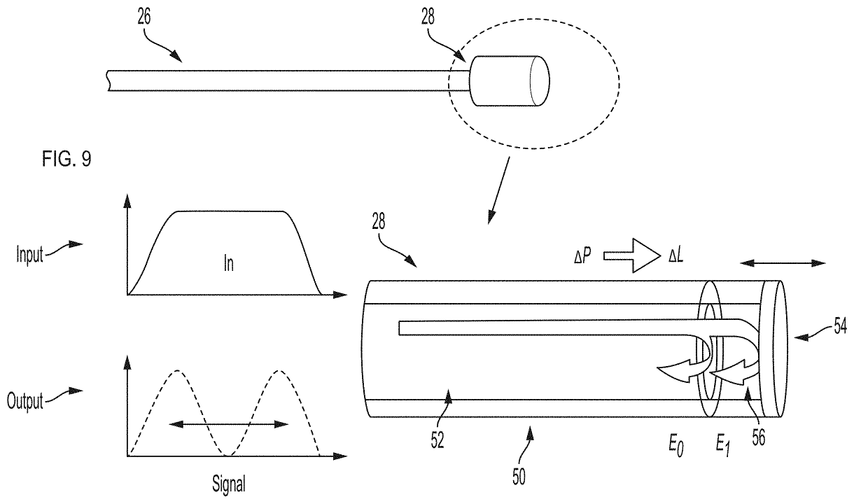

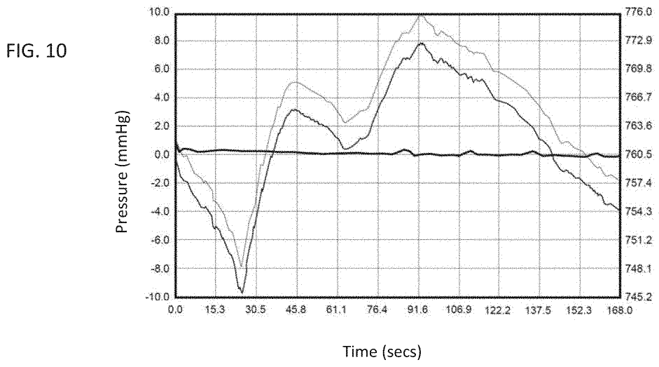

With reference to FIG. 9, the operation of the optical pressure sensor 28 according to one aspect of the present disclosure will be explained. The optical pressure sensor 28 can include a cylindrical housing or capsule 50, a fiber optic core 52, a diaphragm 54, and a gap 56 extending in between the distal end of the fiber optic core 52 and the diaphragm 54. In operation, a light signal (input) is emitted into the fiber optic core 52 via the lead 26, such as via the controller 22 in FIG. 4. Some of the light signal reflects off of the distal end of the fiber optic core (E0) and some of the light reflects off of the diaphragm (E1). The distance between the core 52 and the diaphragm 54 can be measured by the controller 22 based on the phase shift between the reflected parts (E0, E1) of the light signal (Input). With the distance known, any differences in pressure (AP) will cause the diaphragm 54 to deform, which in turn results in changes in the measured phase shift (ΔL). With calibration, these changes in phase shift can be converted into pressure measurements, such as shown (by way of example only) in the chart shown in FIG. 10.

Any of a variety of commercially available and/or specially developed optical pressure sensors may be used with the present disclosure. Optical pressure sensors are advantageous in that they typically are small in size (e.g. 0.125 mm outer diameter), produce no electrical interference, and produce low drift (e.g. 1 mmHg over 7 days). The controller 22 of FIG. 4 may be incorporated into the fluid exchange controller set forth in International Patent Application Publication No. WO/2018/167740, filed Mar. 19, 2018, or may be a separate and stand-alone system.

A computer-implemented method conducted by the control unit 22 is also described herein. In one example, the control unit 22 may include a processor for conducting the steps of this method. The control unit 22 may be initiated to begin delivering fluid through the sensing catheter 10. The fluid may be provided through a tube set attachment 31. After the fluid has begun flowing through the sensing catheter 10, the at least one sensor 28 provided on the sensing catheter 10 will measure a first pressure value of the fluid flowing through the sensing catheter 10 and send this first pressure value signal to the control unit 22. The first pressure value signal includes the first pressure value measurement. The control unit 22 receives this first pressure value signal and logs this signal information for comparison at a later time. The control unit 22 may then be initiated to deliver additional fluid through the sensing catheter 10.

As the additional fluid is being delivered through the sensing catheter 10, the at least one sensor 28 may measure a second pressure value of the fluid flowing through the sensing catheter 10 and send this second pressure value signal to the control unit 22. The second pressure value signal may include the second pressure value measurement. The control unit 22 receives this second pressure value signal and logs this signal information for comparison to the first pressure value. The control unit 22 may then compare the first pressure value of the fluid to the second pressure value of the fluid. In the event a difference between the first pressure value and the second pressure value is below a threshold value, the control unit 22 may be configured to supply additional fluid through the sensing catheter 10. In the event the difference between the first pressure value and the second pressure value is above the threshold value, the control unit 22 may be configured to drain fluid from the sensing catheter 10. This described method may be continuously performed by the control unit 22 as the sensing catheter 10 is used to supply fluid to a patient. For example, the control unit 22 may continuously receive pressure value signals from the at least one sensor 28 and continuously compare the pressure value signals to one another as fluid is directed through the sensing catheter 10. Based on the differences between the measured pressure values, the control unit 22 may continuously monitor the pressure value levels in the sensing catheter 10 and appropriately adjust the supply and drainage of the fluid to/from the sensing catheter 10. It is also contemplated that the at least one sensor 28 may be configured to measure pressure values in the sensing catheter 10 in a predetermined periodic manner. For example, the at least one sensor 28 may be programmed to take pressure value measurements every 5 seconds. It is to be understood, however, that this time period may be altered. The control unit 22 may be used to program the predetermined periodic manner in which the at least one sensor 28 take the pressure value measurements. In another example, the at least one sensor 28 may be used to measure an insertion pressure of the sensing catheter 10 as the sensing catheter 10 is inserted into the patient.

Any of the features or attributes of the above the above described embodiments and variations can be used in combination with any of the other features and attributes of the above described embodiments and variations as desired. From the foregoing disclosure and detailed description of certain preferred embodiments, it is also apparent that various modifications, additions and other alternative embodiments are possible without departing from the true scope and spirit. The embodiments discussed were chosen and described to provide the best illustration of the principles of the present disclosure and its practical application to thereby enable one of ordinary skill in the art to utilize the invention in various embodiments and with various modifications as are suited to the particular use contemplated. All such modifications and variations are within the scope of the present disclosure as determined by any resulting claims when interpreted in accordance with the benefit to which they are fairly, legally, and equitably entitled.

The invention claimed is:

1. A catheter for use in a fluid exchange system, comprising:
an external catheter wall extending between a proximal end and a distal end of the catheter;
a fluid column chamber at a distal region of the catheter, wherein the fluid column chamber comprises a plurality of fluid exchange apertures disposed in the external catheter wall;
a first lumen in fluid communication with the fluid column chamber and adapted for aspirating fluid from the fluid column chamber;
a second lumen in fluid communication with the fluid column chamber and adapted for irrigating fluid into the fluid column chamber; and
a first sensor positioned to measure a fluid pressure within a fluid column of the fluid column chamber, wherein the first sensor is disposed on a lead, the lead being provided within an interior of the catheter or an exterior of the catheter.

2. The catheter of claim 1, wherein the first sensor is configured to gather data that can be used to perform at least one of the following functions: monitor fluid pressure, adjust a supply of the fluid to a patient, or adjust a drainage of the fluid from the patient.

3. The catheter of claim 1, wherein the first sensor is affixed at its distal end to an inner surface of the fluid column chamber.

4. The catheter of claim 1, further comprising:
a second dedicated lumen, wherein a distal end of the second dedicated lumen opens into a sensing channel, wherein the sensing channel is in fluid communication with an area external to the catheter; and
a second sensor positioned to measure a fluid pressure within the sensing channel, wherein the second sensor is positioned on a lead, the lead extending through the second dedicated lumen.

5. The catheter of claim 4, wherein the second sensor measures a fluid pressure external to the catheter and a fluid pressure in the fluid column chamber.

6. The catheter of claim 1, wherein the first sensor is provided on the lead with at least one additional sensor.

7. The catheter of claim 1, further comprising a second sensor provided on an exterior surface of the external catheter wall, and
wherein an aperture is defined in the external catheter wall such that at least a portion of the second sensor is provided within the aperture.

8. The catheter of claim 1, wherein the first sensor is adhesively affixed to the catheter.

9. A fluid exchange system, comprising:
a control unit comprising a processor;
a tube set attachment removably connected to the control unit, the tube set attachment comprising a tube set fluidly connected to a fluid source and a drainage receptacle; and
a catheter fluidly connected to the tube set, the catheter comprising:
an external catheter wall extending between a proximal end and a distal end of the catheter;
a fluid column chamber at a distal region of the catheter, wherein the fluid column chamber comprises a plurality of fluid exchange apertures disposed in the catheter wall;
a first lumen in fluid communication with the fluid column chamber and adapted for aspirating fluid from the fluid column chamber;
a second lumen in fluid communication with the fluid column chamber and adapted for irrigating fluid into the fluid column chamber; and
a first sensor positioned to measure a fluid pressure within a fluid column of the fluid column chamber, wherein the first sensor is disposed on a lead, the lead extending through a first dedicated lumen formed within the catheter or on an exterior of the catheter,
wherein the control unit is configured to supply the fluid to a patient through the tube set and drain the fluid from the patient via the tube set, and
wherein the control unit is configured to receive measurements from the first sensor to monitor fluid pressure, adjust the supply of the fluid to the patient, or adjust the drainage of the fluid from the patient.

10. The fluid exchange system of claim 9, wherein the first sensor is configured to gather data that can be used to perform at least one of the following functions: adjust a supply of the fluid to a patient and adjust a drainage of the fluid from the patient.

11. The fluid exchange system of claim 9, wherein the first sensor is affixed at its distal end to an inner surface of the catheter.

12. The fluid exchange system of claim 10, further comprising:
a second dedicated lumen, wherein a distal end of the second dedicated lumen opens into a sensing channel, wherein the sensing channel is in fluid communication with an area external to the catheter; and
a second sensor positioned to measure a fluid pressure within the sensing channel, wherein the second sensor is positioned on a lead, the lead extending through the second dedicated lumen.

13. The fluid exchange system of claim 9, wherein the first sensor measures a fluid pressure external to the catheter and a fluid pressure in the fluid column chamber.

14. The fluid exchange system of claim 9, wherein the first sensor is provided on the lead along with at least one additional sensor.

15. The fluid exchange system of claim 9, further comprising a second sensor provided on an exterior surface of the external catheter wall, and
wherein an aperture is defined in the external catheter wall such that at least a portion of the second sensor is provided within the aperture.

16. The fluid exchange system of claim 9, wherein the first sensor is adhesively affixed to the catheter.

17. A computer-implemented method of using the fluid exchange system of claim 1, the method comprising:
initiating, using a processor, a control unit to deliver a fluid through the second lumen;
receiving, at the processor, a first pressure value of the fluid, wherein the first pressure value is measured by the first sensor;
initiating, using the processor, the control unit to deliver additional fluid through the second lumen;
receiving, at the processor, a second pressure value of the fluid, wherein the second pressure value is measured by the first sensor;
comparing, using the processor, the first pressure value and the second pressure value; and
in an event a difference between the first and second pressure values exceeds a pressure threshold value, initiating, using the processor, the control unit to drain the fluid through the first lumen, and, in the event the difference between the first and second pressure values is less than the pressure threshold value, initiating, using the processor, the control unit to deliver additional fluid through the second lumen.

18. The computer-implemented method of claim 17, wherein the first pressure value and the second pressure value are received from the first sensor provided at at least one of an exterior surface of the catheter and a position in the fluid column chamber defined in the catheter.

19. The computer-implemented method of claim 17, wherein the first pressure value and the second pressure value are received from the first sensor provided at an exterior surface of the catheter and a second sensor provided at a position in the fluid column chamber defined in the catheter.

20. The computer-implemented method of claim 17, further comprising detecting, using the processor, an insertion pressure value for the catheter using the first sensor.

21. A catheter for use in a fluid exchange system, comprising:
an external catheter wall extending between a proximal end and a distal end of the catheter;
a fluid column chamber provided within the catheter, wherein the fluid column chamber comprises a plurality of fluid exchange apertures disposed in the external catheter wall;
a first lumen in fluid communication with the fluid column chamber and adapted for aspirating fluid from the fluid column chamber;
a second lumen in fluid communication with the fluid column chamber and adapted for irrigating fluid into the fluid column chamber; and
a lead positioned either internally or externally to the catheter, the lead comprising a plurality of sensors positioned in intervals along the length of the lead, wherein the sensors are configured to measure a fluid pressure either within the fluid column chamber or external to the catheter to identify a fluid pressure gradient along a length of at least a portion of the catheter.
